(19) United States
(12) Patent Application Publication
(10) Pub. No.: US 2014/0242145 A1
(43) Pub. Date: Aug. 28, 2014

(54) CHITOSAN NANOFIBER FOR ANIONIC PROTEIN DRUG DELIVERY, METHOD OF PREPARING THE SAME, AND PHARMACEUTICAL PREPARATION FOR TRANSMUCOSAL ADMINISTRATION COMPRISING THE CHITOSAN NANOFIBER

(71) Applicant: KNU-INDUSTRY COOPERATION FOUNDATION, Chuncheon-si (KR)

(72) Inventors: Hyuk Sang YOO, Seoul (KR); Ji Suk CHOI, Chuncheon-si (KR); Younghee KIM, Gangneung-si (KR); Jihyun KANG, Yangyang-gun (KR)

(73) Assignee: KNU-INDUSTRY COOPERATION FOUNDATION, Chuncheon-si (KR)

(21) Appl. No.: 14/146,252

(22) Filed: Jan. 2, 2014

(30) Foreign Application Priority Data
Feb. 28, 2013 (KR) 10-2013-0022127

(51) Int. Cl. A61K 9/70; A61K 47/36; A61K 38/38

(52) U.S. Cl. CPC A61K 9/70; A61K 38/385; A61K 47/36
USPC 424/443; 514/15.2; 264/465

(57) ABSTRACT

A chitosan nanofiber for delivering an anionic protein drug, a method of preparing the same, and a pharmaceutical preparation for transmucosal administration including the chitosan nanofiber are provided. The chitosan nanofiber including an anionic protein drug in a core and chitosan in a shell is prepared by coaxial electrospinning an aqueous solution of the anionic protein drug through an inner nozzle and a solution of the chitosan or a chitosan derivative through an outer nozzle.

### RELATED APPLICATIONS

This application claims the benefit of Korean Patent Application No. 10-2013-0022127, filed on Feb. 28, 2013, in the Korean Intellectual Property Office, the disclosure of which is incorporated herein in its entirety by reference.

### BACKGROUND

1. Field

The present disclosure relates to a chitosan nanofiber for anionic protein drug delivery, a method of preparing the same, and a pharmaceutical preparation for transmucosal administration including the chitosan nanofiber, and more particularly, to a chitosan nanofiber for anionic protein drug delivery that is prepared by coaxial electrospinning, a method of preparing the same, and a pharmaceutical preparation for transmucosal administration including the chitosan nanofiber.

2. Description of the Related Art

Protein drugs may be delivered to the human body by being entrapped in microspheres or bound to polymer. However, when such a form of protein drug is orally administered, drug delivery efficiency in the body may be very low due to a hepatic and gastric first-pass effect. To address this drawback, using water-in-oil-in-water (w/o/w) double emulsions or liposomes has been suggested. However, they require complicated preparation processes, are vulnerable to protein denaturation, and are not physically strong enough to protect the protein drug from damage. A protein drug may also be mucosally administered via direct injection into blood vessels for higher delivery efficiency in the body. However, this method may cause aversion in patients due to fear or pain from syringe needles. Moreover, due to severe loss of the injected protein drug while circulating blood vessels, a large amount of the protein drug should be injected, which is economically unfeasible. Therefore, there is a need for efficient drug delivery systems for increased absorption of the protein drug in the body.

An oral-mucosal drug delivery method has been suggested as a method for increasing the systemic absorption of protein drugs. Oral mucosa with well-developed blood vessels is effective for delivery of drugs such as protein and enzyme activity thereof is low. Oral mucosa is also more tolerable of allergens than other mucosal tissue, and is suitable for topical and systemic administration of drugs. Most of all, oral-mucosal administration of protein drugs may avoid a hepatic and gastric first-pass effect, thus reducing drug loss. For these reasons, recently there has been increased research into systemic administration of protein drugs through oral mucosa.

Nanofibers have been suggested as a preparation for increasing the systemic absorption of protein drugs. Nanofibers may be prepared by electrospinning, in which a droplet of a polymer solution on the end of a needle is stretched in the form of a stream of polymer fluid by applying a voltage higher than the surface tension of the droplet of the polymer solution onto the droplet of the polymer solution, and is collected as nanofibers on a collector. The polymer used in preparing nanofibers may be polyvinylpyrolidone, polycarprolactone, or poloxamer. Such prepared nanofibers may have a porous structure as a stack of entangled nanosized fibers with a large surface area relative to volume. Accordingly, the porous structure of the nanofibers is able to trap a material such as a drug therein and may be easily adhered even to cutaneous mucosa. For nanofibers prepared by electrospinning through a single nozzle, a drug entrapped by such nanofibers may be so rapidly released at an early stage, thus causing a side effect from the abrupt release of a high dose of the drug. Mostly, an organic solvent is used in the polymer solution for electrospinning. However, in preparing the polymer solution for electrospinning through a single nozzle, a drug substance such as protein may be exposed to the organic solvent for so long to be denatured, thus losing activity at this period. Furthermore, uniformly dispersing a polymer together with a protein drug as an aqueous solution is not easy, consequently hindering the entrapping of the protein drug in nanofibers.

As an alternative to this drawback, nanofibers may be prepared by coaxial electrospinning, in which core-shell structured nanofibers are prepared by applying a voltage higher than the surface tension of an electrospinning solution onto tips of a syringe with two coaxially aligned nozzles as illustrated in FIG. 1 so that the core-shell structured nanofibers are piled on a collector. Normally, an electrospinning solution for the core may be water, and one for the shell may be an organic solvent. The use of the two types of solvents with different properties may cause a core-shell structure to be formed through phase separation during the electrospinning. Since the protein drug is dispersed in water, not an organic solvent, it is unlikely to be denatured during the electrospinning. Furthermore, the core-shell structured nanofibers prepared by coaxial electrospinning is made in the form of the protein drug being located inside a polymer shell, and thus may prevent abrupt release of a large amount of the drug at an early stage (Kangjie Zhu et al., Modulation of Protein Release from Biodegradable Core-Shell Structured Fibers Prepared by Coaxial Electrospinning, Journal of Biomedical Materials Research Part B: Applied Biomaterials, 2005, 50-57).

However, conventional nanofibers with core of a protein drug prepared by coaxial electrospinning have a low encapsulation efficiency of about 8.3% of protein drugs (F. Z. Cui et al., Electrospun collagen-chitosan nanofiber: A biomimetic extracellular matrix for endothelial cell and smooth muscle cell, Acta Biomaterialia, 2010(6), 372-382), and the adhesion thereof to mucosa is not so strong.

### SUMMARY

Provided are nanofibers for delivering an anionic protein drug without burst release of the anionic protein drug, the nanofibers having a high drug encapsulation efficiency and good adhesion to oral mucosa.

Provided are methods of preparing the nanofibers for anionic protein drug delivery.

Provided are pharmaceutical preparations for transmucosal administration for anionic protein drug delivery, including the nanofibers for anionic protein drug delivery.

Additional aspects will be set forth in part in the description which follows and, in part, will be apparent from the description, or may be learned by practice of the presented embodiments.

According to an aspect of the present invention, a chitosan nanofiber for delivering an anionic protein drug includes the anionic protein drug in a core and chitosan in a shell, which is obtained by coaxial electrospinning an aqueous solution of the anionic protein drug through an inner nozzle and a solution of the chitosan or a chitosan derivative through an outer nozzle.

According to another aspect of the present invention, a method of preparing the above-described chitosan nanofiber includes: injecting the aqueous solution of the anionic protein drug into the inner nozzle of a dual nozzle; injecting the solution of the chitosan or the chitosan derivative into the outer nozzle of the dual nozzle; and performing coaxial spinning at a flow rate of about 0.05 to 0.1 mL/h at an inner nozzle and a flow rate of about 0.5 to 1.5 mL/h at an outer nozzle, while applying a voltage of about 20 kV to about 27 kV to prepare the chitosan nanofiber.

According to another aspect of the present invention, a pharmaceutical preparation for transmucosal administration of an anionic protein drug includes the above-described the chitosan nanofiber including the anionic protein drug in a core and chitosan in a shell.

### BRIEF DESCRIPTION OF THE DRAWINGS

These and/or other aspects will become apparent and more readily appreciated from the following description of the embodiments, taken in conjunction with the accompanying drawings in which:

FIG. 1 is a view illustrating an experimental device for preparing a core-shell structure of a nanofiber by coaxial electrospinning;

FIG. 2 is a schematic view of a chitosan nanofiber, according to an embodiment of present disclosure, prepared by coaxial electrospinning, including a magnified schematic cross-sectional view thereof;

FIG. 3 illustrates field emission scanning electron microscopic (FESEM) images of a chitosan nanofiber of Example 1 at different magnifications before and after neutralization;

FIG. 4 illustrates optical microscopic images of chitosan nanofibers prepared by coaxial electrospinning at different flow rates at an inner nozzle and a fixed flow rate at an outer nozzle and a chitosan nanofiber prepared by electrospinning through a single nozzle;

FIG. 5 illustrates confocal laser microscopic images illustrating encapsulation patterns of fluorescein 5(6)-isothiocyanate labeled bovine serum albumin (FITC-BSA) in chitosan nanofibers prepared by coaxial electrospinning at different flow rates at an inner nozzle and at a fixed flow rate at an outer nozzle; and

FIG. 6 is a standard fluorescent curve of FITC-BSA in distilled water with respect to the concentration of the FITC-BSA solution.

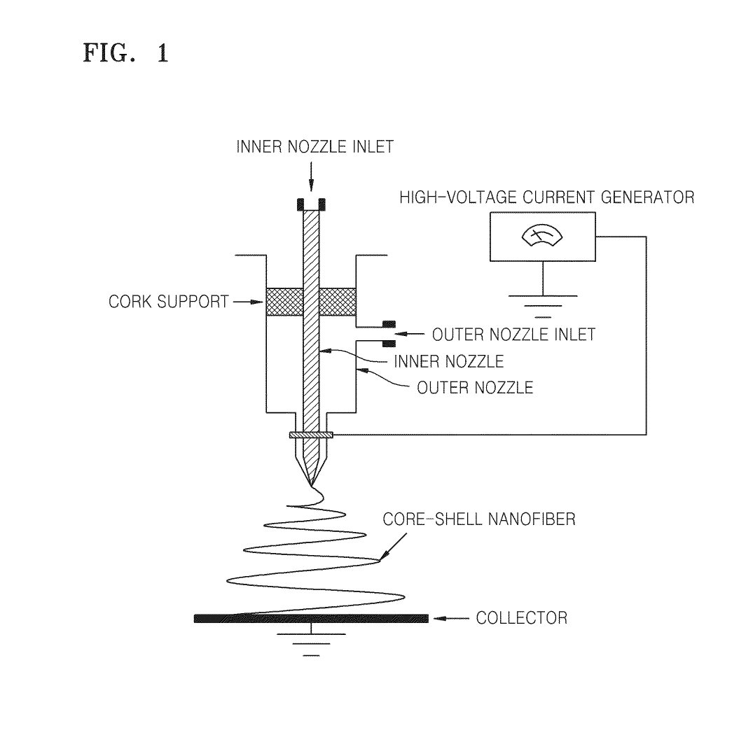

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Unless otherwise defined, all terms (including technical and scientific terms) used herein have the same meaning as commonly understood by one of ordinary skill in the art to which this invention belongs. Although a few embodiments of the present disclosure have been shown and described, it would be appreciated by one of ordinary skill in the art that changes may be made in these exemplary embodiments without departing from the principles and spirit of the invention, the scope of which is defined in the claims and their equivalents. The disclosures of any references referred to herein are incorporated herein in their entirety by reference.

As a result of research into nanofiber preparation for transmucosal administration that may efficiently entrap an anionic protein drug and facilitate adhesion of the anionic protein drug to mucosa for improved protein drug delivery, the inventors of the present disclosure found that a chitosan nanofiber prepared by coaxial electrospinning to have a shell of polymeric chitosan or a derivative thereof and a core of an anionic protein drug may satisfy the goal of the research.

According to an embodiment of the present disclosure, provided is a chitosan nanofiber for delivering an anionic protein drug, the chitosan nanofiber including the anionic protein drug in a core and chitosan in a shell, which is obtained by coaxial electrospinning an aqueous solution of the anionic protein drug through an inner nozzle and a solution of the chitosan or a chitosan derivative through an outer nozzle.

The aqueous solution of the anionic protein drug may further include a viscosity modifier selected from the group consisting of polyvinyl alcohol, polybutene, sodium polyacrylate, povidone, polyphosphorylcholine glycol acrylate, xanthan gum, guar gum, gelatin, methylcellulose, hycel, carbomer, and a combination thereof. The use of the modifier may prevent generation of beads, not perfect fibrous form, in coaxial electrospinning for forming nanofibers, which may occur from a low viscosity of the aqueous solution added to an inner nozzle for forming a core.

The anionic protein drug may be any anionic protein drugs that are administered topically or systemically into the mucosa of the human body. The anionic protein drug may be selected from the group consisting of human growth hormone, bovine serum albumin (BSA), mussel adhesive protein, and a combination thereof, but is not limited thereto.

The chitosan derivative may be acrylated chitosan, thiolated chitosan, or phosphorylated chitosan, but is not limited thereto. For example, any chitosan derivatives able to form a chitosan nanofiber by coaxial electrospinning may be used.

Chitosan is a copolymer of glucosamine and N-acetylglocosamine obtained by deacetylation of chitin that is abundant in crustacean shells. Chitosan, which has recently been approved by the US FDA for medical or food use, also has high biocompatibility. Chitosan costs low because it is obtained from food waste such as crab or shrimp shells, and chitosan may be decomposable by chitinase secreted by intestinal bacteria in the human body. For example, the chitosan or chitosan derivative may have a molecular weight of about 70K to about 500K, and in some embodiments, about 50K to about 300K, and in some other embodiments, about 100K. When the molecular weight of the chitosan is out of these ranges, it may be unsuccessful to form the chitosan nanofiber due to generation of beads hindering the retention of a fibrous shape.

According to another embodiment of the present invention, provided is a method of preparing a chitosan nanofiber according to any of the above-described embodiments including: injecting the aqueous solution of the anionic protein drug into the inner nozzle of a dual nozzle; injecting the solution of the chitosan or the chitosan derivative into the outer nozzle of the dual nozzle; and performing coaxial spinning at a flow rate of about 0.05 mL/h to about 0.1 mL/h for an inner nozzle and a flow rate of about 0.5 mL/h to about 1.5 mL/h for an outer nozzle, while applying a voltage of about 20 kV to about 27 kV to prepare the chitosan nanofiber.

The aqueous solution of the anionic protein drug that is used in the method may further include a viscosity modifier selected from the group consisting of polyvinyl alcohol, polybutene, sodium polyacrylate, povidone, polyphosphorylcholine glycol acrylate, xanthan gum, guar gum, gelatin, methylcellulose, hycel, carbomer, and a combination thereof. In the aqueous solution of the anionic protein drug, a concentration of the anionic protein drug may be from about 0.001 g/mL to about 0.007 g/mL, and a concentration of the viscosity control agent may be from about 0.005 g/mL to about 0.05 g/mL. When the concentrations of the anionic protein drug or the viscosity control agent are out of these ranges, the amount of the anionic protein drug entrapped in the chitosan nanofiber may be small, or the concentration of the aqueous solution to be discharged through the inner nozzle may not be appropriate for forming the chitosan nanofiber.

A solvent of the solution of the chitosan or the chitosan derivative may be an acidic solution, for example, a mixed solvent of trifluoroacetic acid and dichloromethane, or a mixed solvent of hexafluoroisopropanol and trifluoroacetic acid. A concentration of the solution including the chitosan or the chitosan derivative may be from about 0.05 g/mL to about 0.1 g/mL. When the concentration of the solution including the chitosan or the chitosan derivative is out of this range, the viscosity thereof may be low and thus cause generation of beads, or the viscosity thereof may be high and thus cause sparking during the electrospinning due to unstable current flow.

For example, the flow rate for the inner nozzle may be 0.07 mL/h to about 0.09 mL/h, and the flow rate for the outer nozzle may be from about 0.9 mL/h to about 1.1 mL/h.

In the chitosan nanofiber prepared by the above-described method, an amine group of the chitosan may form a polar salt (—NH3+CF3COO—) with trifluoroacetic acid (TFA) in the solvent, and thus, the chitosan nanofiber may be highly soluble in water. To prevent this, the method may further include neutralizing the chitosan nanofiber in an alkaline solution after the coaxial electrospinning.

The alkaline solution may be an aqueous solution, such as a sodium carbonate solution, a calcium carbonate solution, a potassium carbonate solution, or a sodium phosphate solution. A concentration of the alkaline solution may be from about 1M to about 6M. The neutralizing may be performed by immersing the chitosan nanofiber in such an alkaline solution for about 6 to 24 hours. A diameter of the chitosan nanofiber may swell during the neutralizing, while a shape of the chitosan nanofiber may remain constant.

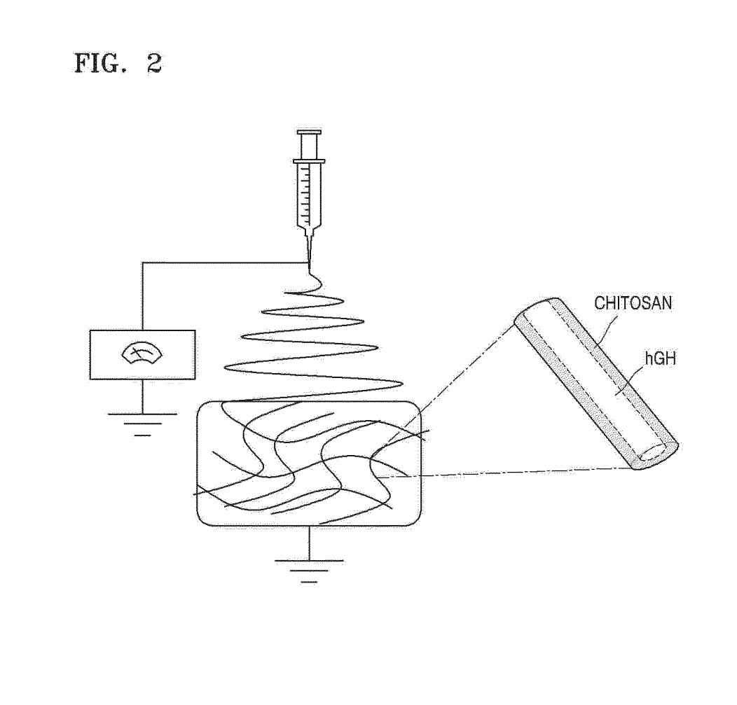

In the chitosan nanofiber according to the present disclosure, the anionic protein drug is in the core, which is covered by the shell including the chitosan or chitosan derivative. FIG. 2 is a magnified perspective view of a chitosan nanofiber, according to an embodiment of the present disclosure, prepared by coaxial electrospinning using human growth hormone (hGH) as an anionic protein drug. Referring to FIG. 2, a cross-section of the chitosan nanofiber prepared by coaxial electrospinning and collected by a collector electrode shows that a core region, including hGH as an anionic protein drug, is covered by a shell region including chitosan.

Chitosan has a cationic amine group. Accordingly, the chitosan nanofiber according to the present disclosure may efficiently entrap an anionic drug.

In addition, due to the shell region including cationic chitosan, the chitosan nanofiber may have strong adhesion to anionic biological mucosa. Accordingly, the chitosan nanofiber according to the present disclosure may facilitate delivery of an anionic protein drug through oral mucosa. The chitosan nanofiber according to the present disclosure may ensure sustained release of the anionic protein drug, thus suppressing abrupt release, since it includes the anionic protein drug in the core.

According to another embodiment of the present disclosure, provided is a pharmaceutical preparation for transmucosal administration for delivering an anionic protein drug including any of the chitosan nanofibers according to the above-described embodiments of the present disclosure.

The pharmaceutical preparation for transmucosal administration may be administered via any biological mucosa for drug administration, for example, oral mucosa, ocular mucosa, nasal mucosa, ear mucosa, gastric mucosa, vaginal mucosa, or anal mucosa. For example, the pharmaceutical preparation for transmucosal administration may be administered through oral mucosa. As described above, oral mucosa with well-developed blood vessels may be effective for protein drug delivery. Oral-mucosal administration of a protein drug may avoid a gastric or hepatic first-pass effect, thus reducing drug loss.

In some embodiments, the chitosan nanofiber may be used as a pharmaceutical preparation for transmucosal administration as it is or via a conventional formulation process known in the art.

One or more embodiments of the present invention will now be described in detail with reference to the following examples. However, these examples are not intended to limit the scope of the one or more embodiments of the present invention.

### EXAMPLE 1

### Preparation of Chitosan/FITC-BSA Nanofiber by Coaxial Electrospinning

A chitosan/FITC-BSA nanofiber, including chitosan (Mw=100,000, Wako) in a shell and bovine serum albumin (BSA) (Mw=66,000, Sigma) labeled with fluorescein 5(6)-isothiocyanate (FITC) (Mw=389.38, Sigma), and polyvinyl alcohol (PVA) (Mw 27,000, Sigma) in a core, was prepared as follows: A chitosan solution to be discharged through an outer nozzle was prepared in a concentration of about 7% (w/v) in a mixed solvent of TFA and dichloromethane (DCM) (7:3, v/v). A solution to be discharged through an inner nozzle was prepared by dissolving 0.35 mg/mL of FITC-BSA in a 1% (w/v) PVA solution. The FITC-BSA/PVA solution and the chitosan solution were supplied at a flow rate of about 0.08 mL/h and a flow rate of about 1.0 mL/h through the inner and outer nozzles of a dual nozzle, respectively (hereinafter, simply referred to as “flow rates of 0.08-1.0 mL/h at the inner and outer nozzles”), while applying a voltage of about 25 kV. A distance between the nozzle and the ground collector electrode was maintained at about 15 cm.

### EXAMPLE 2

### Neutralization of Chitosan/FITC-BSA Nanofiber

To neutralize the chitosan/FITC-BSA nanofiber with cations originating from the amine group of chitosan, the chitosan/FITC-BSA nanofiber was immersed in a 4M sodium carbonate solution at a temperature of about 37° C. for about 12 hours, washed ten times with distilled water, and then freeze-dried.

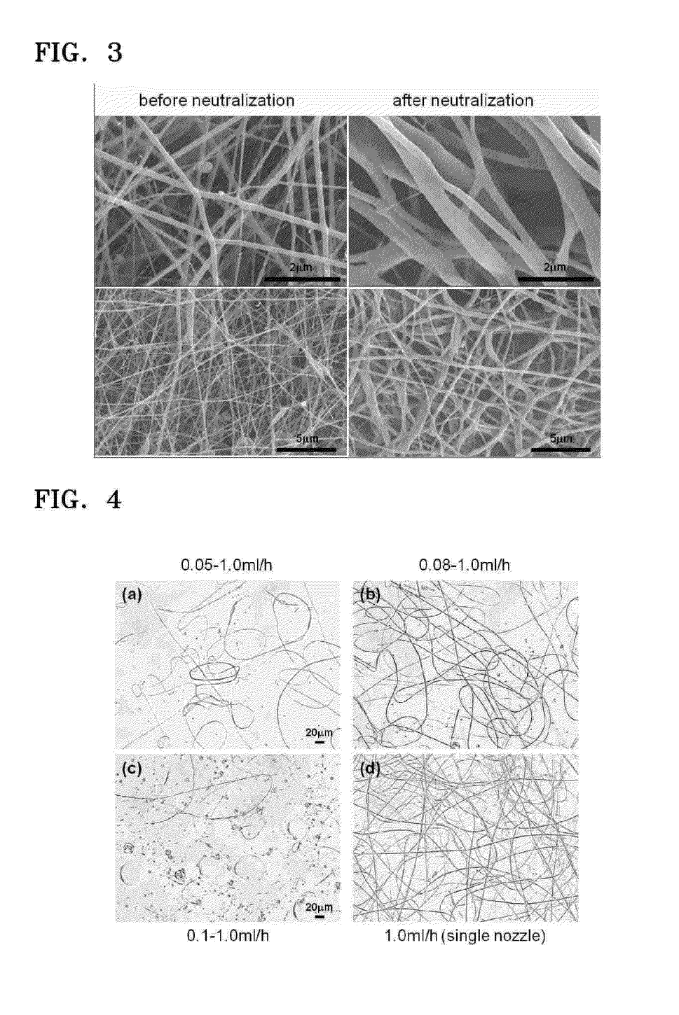

Shapes of the chitosan/FITC-BSA nanofiber before and after the neutralization were observed using a field emission scanning electron microscope (FESEM). The results are shown in FIG. 3.

FIG. 3 illustrates FESEM images of the chitosan/FITC-BSA nanofiber of according to an example of the present invention at different magnifications before and after the neutralization.

The chitosan/FITC-BSA nanofiber has a diameter of about 122.6±32.5 nm and about 252.5±98.4 nm before and after the neutralization, respectively. The diameter after the neutralization was almost twice of that before the neutralization, which is attributed to swelling during the neutralization with the sodium carbonate solution. However, the shape of the chitosan/FITC-BSA nanofiber remained constant during the neutralization.

### EXPERIMENTAL EXAMPLE 1

### Effects of Flow Rate on the Formation of Chitosan/FITC-BSA Nanofiber

Chitosan/FITC-BSA nanofibers were prepared in the same manner as in Example 1, except that the flow rate of the FITC-BSA/PVA solution at the inner nozzle and that of the chitosan solution at the outer nozzle were about 0.05-1.0 mL/h and about 0.1-1.0 mL/h.

A chitosan/FITC-BSA nanofiber was prepared by electrospinning through a single nozzle. In particular, an electrospinning solution was prepared by dissolving 0.7g/mL of chitosan in a mixed solvent of TFA and DCM (7:3, v/v) and adding 0.0035 g/mL of BSA and 0.01 g/mL of a viscosity modifier (polyvinyl alcohol) to the solution. This solution was supplied at a flow rate of about 0.8 mL/h through the single nozzle while applying a voltage of about 25 kV. A distance between the nozzle and the ground electrode was maintained at about 10 cm.

Shapes of the chitosan/FITC-BSA nanofiber of Example 1 and the chitosan/FITC-BSA nanofibers prepared while varying the flow rates at the inner and outer nozzles or using a single nozzle as described above were observed using an optical microscope. The results are shown in FIG. 4.

The chitosan/FITC-BSA nanofiber prepared by electrospinning the 7% (w/v) chitosan solution at a flow rate of 1.0 mL/h through a single nozzle while applying a voltage of about 25kV had a constant shape (see FIG. 4(d)). Meanwhile, the chitosan/FITC-BSA nanofiber prepared by coaxial electrospinning through the dual nozzle was found to include beads generated due to low volatility of water-based inner solution (see FIG. 4(c)). A chitosan/FITC-BSA nanofiber with optimal characteristics among the chitosan/FITC-BSA nanofibers prepared at a fixed flow rate at the outer nozzle and a varying flow rate at the inner nozzle was obtained at flow rates of 0.08-1.0 mL/h at the inner and outer nozzles (see FIG. 4(b)).

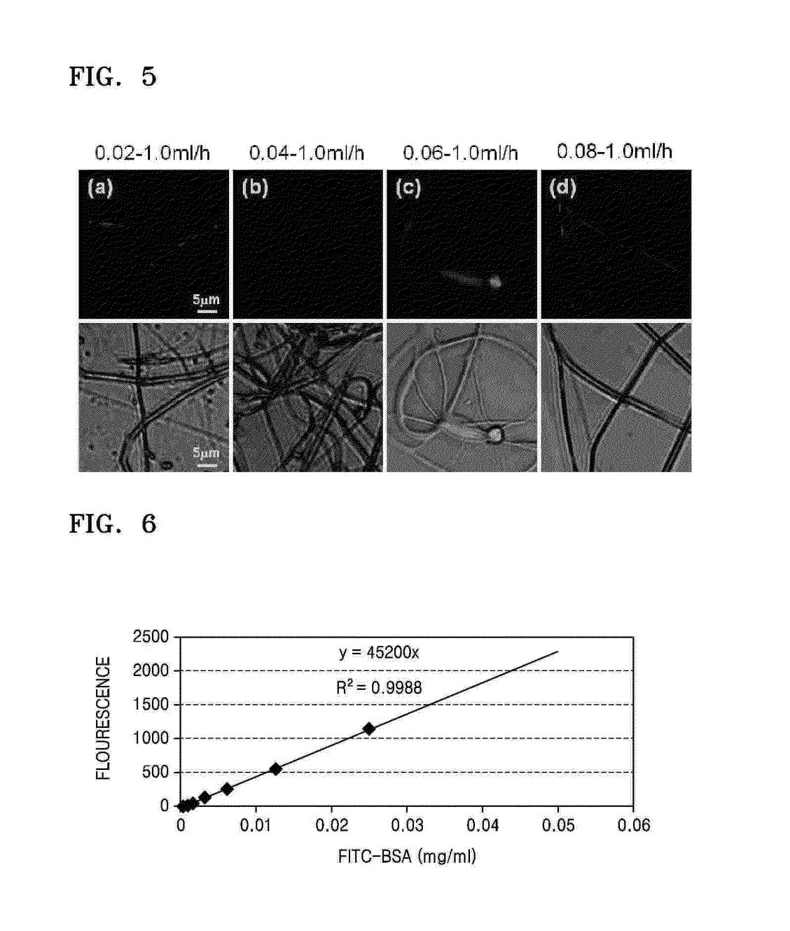

Chitosan/FITC-BSA nanofibers were prepared in the same manner as in Example 1, except that the flow rate of the FITC-BSA/PVA solution at the inner nozzle and that of the chitosan solution at the outer nozzle were varied to 0.02-1.0 mL/h, 0.04-1.0 mL/h, and 0.06-1.0 mL/h. The encapsulation patterns of FITC-BSA in the chitosan/FITC-BSA nanofiber of Example 1 and the chitosan/FITC-BSA nanofiber samples prepared by varying the flow rate at the inner nozzle and at a fixed flow rate at the outer nozzle were observed using a confocal laser microscope. The results are shown in FIG. 5 (at a 600×-magnification, inner-outer).

Referring to FIG. 5, in the chitosan/FITC-BSA nanofibers prepared at a flow rate of 0.02 mL/h and 0.04 mL/h at the inner nozzle, only some of the chitosan nanofibers were found to entrap FITC-BSA (see FIGS. 5(a) and 5(b)). The chitosan/FITC-BSA nanofiber prepared at a flow rate of 0.06 mL/h was found to entrap FITC-BSA only around beads due to generation of the beads. Meanwhile, in the chitosan/FITC-BSA nanofiber of Example 1 prepared by coaxial electrospinning at flow rates of 0.08-1.0 mL/h at the inner and outer nozzles, uniform distribution of FITC-BSA in most of the chitosan nanofibers was observed (see FIG. 5(d)).

### EXPERIMENTAL EXAMPLE 2

### Measurement of Encapsulation Efficiency of FITC-BSA

To measure the encapsulation efficiency of BSA in the core of each of the chitosan/FITC-BSA nanofibers of Examples 1 and 2, after weighing the chitosan/FITC-BSA nanofiber before neutralization, it was completely dissolved in 1 mL of distilled water and then the fluorescence was measured by a fluorescence spectrometer (excitation at 495 nm and emission at 520 nm). FIG. 6 is a standard fluorescence curve of FITC-BSA, obtained using FITC-BSA solutions in different concentrations (50 mg/mL, 25 mg/mL, 12.5 mg/mL, 6.25 mg/mL, 3.13 mg/mL, 1.56 mg/mL, 0.78 mg/mL, and 0.39 mg/mL) dissolved in distilled water.

An encapsulation efficiency of FITC-BSA in each chitosan/FITC-BSA nanofiber sample was calculated using the following equation. The results are shown in Table 1 below.

Encapsulation Efficiency (%)=(Actual measured amount of FITC-BSA/Theoretical amount of included FITC-BSA)×100

TABLE 1
Encapsulation efficiencies of FITC-BSA in chitosan/FITC-BSA
nanofibers

| Chitosan/ | | | FITC- | |
|---|---|---|---|---|
| FITC-BSA | Concentration | | BSA | Encapsulation |
| nanofiber | (mg/mL) | Fluorescence | (mg/mL) | efficiency (%) |
| 1 | 2.4 | 320.3 | 0.0065 | 69.6 |
| 2 | 2.1 | 310.9 | 0.0063 | 77.2 |
| 3 | 2.3 | 347.6 | 0.0071 | 78.8 |
| 4 | 2.4 | 377.6 | 0.0077 | 82.0 |
| 5 | 3 | 517.7 | 0.0105 | 89.9 |
| 6 | 4 | 591.8 | 0.0120 | 77.1 |
| | | | Means | 79.1 |
| | | | Standard | 6.7 |
| | | | deviation | |

According to the results in Table 1 above, the chitosan/FITC-BSA nanofibers prepared according to the present disclosure were found to have a high encapsulation efficiency of about 79.1% on average for the anionic protein drug in the chitosan nanofiber. Therefore, the chitosan/FITC-BSA nanofibers according to the embodiments of the present disclosure may efficiently include an anionic protein drug, and thus are economically feasible.

As described above, according to the one or more of the above embodiments of the present invention, a chitosan nanofiber prepared by coaxial electrospinning may have a core-shell structure able to efficiently include an anionic protein drug, in which the anionic protein drug is present in a core region enclosed by a shell region including chitosan or a chitosan derivative thereof. The presence of chitosan or chitosan derivative in the shell region may improve adhesion to mucosa, and thus provide more effective delivery of the protein drug to the body. The presence of the protein drug in the core region may prevent abrupt release of the protein drug, and rather may ensure sustained release of the protein drug in the body, thus preventing a side effect from the abrupt release of a high dose of the protein drug.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

What is claimed is:

1. A chitosan nanofiber for delivering an anionic protein drug, the chitosan nanofiber comprising the anionic protein drug in a core and chitosan in a shell, which is obtained by coaxial electrospinning an aqueous solution of the anionic protein drug through an inner nozzle and a solution of the chitosan or a chitosan derivative through an outer nozzle.

2. The chitosan nanofiber of claim 1, wherein the aqueous) solution of the anionic protein drug further comprises a viscosity modifier selected from the group consisting of polyvinyl alcohol, polybutene, sodium polyacrylate, povidone, polyphosphorylcholine glycol acrylate, xanthan gum, guar gum, gelatin, methylcellulose, hycel, carbomer, and a combination thereof.

3. The chitosan nanofiber of claim 1, wherein the anionic protein drug is selected from the group consisting of human growth hormone (hGH), bovine serum albumin (BSA), mussel adhesive protein, and any combinations thereof.

4. The chitosan nanofiber of claim 1, wherein the chitosan derivative is acrylated chitosan, thiolated chitosan, or phosphorylated chitosan.

5. A method of preparing the chitosan nanofiber of claim 1, the method comprising:
injecting the aqueous solution of the anionic protein drug into the inner nozzle of a dual nozzle;
injecting the solution of the chitosan or the chitosan derivative into the outer nozzle of the dual nozzle; and
performing coaxial spinning at a flow rate of about 0.05 to 0.1 mL/h at an inner nozzle and a flow rate of about 0.5 to 1.5 mL/h at an outer nozzle, while applying a voltage of about 20 kV to about 27 kV to prepare the chitosan nanofiber.

6. The method of claim 5, wherein the aqueous solution of the anionic protein drug further comprises a viscosity control agent selected from the group consisting of polyvinyl alcohol, polybutene, sodium polyacrylate, povidone, polyphosphorylcholine glycol acrylate, xanthan gum, guar gum, gelatin, methylcellulose, hycel, carbomer, and any combinations thereof.

7. The method of claim 5, wherein a solvent of the solution of the chitosan or the chitosan derivative is a mixture of trifluoroacetic acid and dichloromethane, or a mixture of hexafluoroisopropanol and trifluoroacetic acid.

8. The method of claim 5, further comprising neutralizing the prepared chitosan nanofiber in an alkaline solution.

9. The method of claim 8, wherein the alkaline solution is a sodium carbonate solution, a calcium carbonate solution, a potassium carbonate solution, or a sodium phosphate solution.

10. A pharmaceutical preparation for transmucosal administration of an anionic protein drug, the pharmaceutical preparation comprising the chitosan nanofiber of claim 1.

11. The pharmaceutical preparation of claim 10, wherein the pharmaceutical preparation delivers the anionic protein drug via oral mucosa.
